(19) United States
(12) Patent Application Publication
(10) Pub. No.: US 2012/0170710 A1
(43) Pub. Date: Jul. 5, 2012

(54) PATIENT SUPPORT SYSTEM AND SUPPORT SURFACE THEREFOR AND METHOD OF INSTALLATION THEREOF

(76) Inventors: James H. Niedzielski, Fenton, MI (US); Gary E. Terrill, Leroy, MI (US); Brian J. Knoch, Fenton, MI (US)

(21) Appl. No.: 13/290,542

(22) Filed: Nov. 7, 2011

Related U.S. Application Data
(60) Provisional application No. 61/410,502, filed on Nov. 5, 2010.

(51) Int. Cl. A61B 6/03; H05K 13/00; A61B 6/04

(52) U.S. Cl. 378/20; 5/601; 29/592.1

(57) ABSTRACT

A patient support surface, patient support system in combination with a CT scanner, and method of installing a patient support system for a surgical procedure is provided. The patient support surface includes a radiolucent elongate first member having laterally spaced first side members extending between opposite ends with a plurality of cross members extending between the first side members. The support surface further includes an elongate second member having laterally spaced second side members extending between opposite ends with a plurality of cross members extending between the second side members. One of the opposite ends of the first member is configured for attachment to one of the ends of the second member. The other of the opposite ends of the first member is configured for attachment to a first support, and the other of the opposite ends of the second member is configured for attachment to a second support.

### CROSS-REFERENCE TO RELATED APPLICATION

This application claims the benefit of U.S. Provisional Application Ser. No. 61/410,502, filed Nov. 5, 2010, which is incorporated herein by reference in its entirety.

### BACKGROUND OF THE INVENTION

1. Technical Field

This invention relates generally to surgical tables, and more particularly to surgical tables used in conjunction with computed axial tomography (CAT or CT) devices during surgery.

2. Related Art

It is known to perform a surgical procedure on a patient supported on a table while the table extends partially through an annular opening of CT scanner. For example, spinal surgery is typically performed using such a table. The table is typically cantilevered from a single support base at one end, with the free unsupported end being oriented to extend partially through the annular opening of the CT scanner. Although these tables are useful, they have drawbacks. For example, with the table being cantilevered, the table has a patient support portion of a limited length. In addition to the limited length drawback, the support base supporting the table occupies significant space at the supported end. As such, an average size adult patient cannot be fully imaged due to the CT scanner not being able to pass about the support base, thereby interfering with the support base. Further yet, in addition to the aforementioned drawbacks, being cantilevered causes the table to deflect under the load of the patient to varying degrees, depending on the size of the patient, and thus, it can compromise the surgeon's ability to orient the patient to the position desired, particularly if the patient is large. In addition to these drawbacks, the known patient support tables are typically used in combination with support members, such as are commonly used to lift a patient's chest or abdominal region from the table to better position the spine for surgery. However, in doing this, the radially extending space occupied by the table, support member and patient can make it impossible to move the raised portion of the patient through the annular opening of the CT scanner.

Accordingly, there is a need for a patient support system that overcomes the aforementioned drawbacks encountered with known patient support tables used in combination with a CT scanner, as well as overcoming other drawbacks that are encountered by surgeons performing such surgical procedures.

### SUMMARY OF THE INVENTION

In accordance with one aspect of the invention, a patient support surface is provided. The patient support surface includes a radiolucent elongate first member having laterally spaced first side members extending along a length between opposite ends with a plurality of cross members extending between the first side members. The support surface further includes an elongate second member having laterally spaced second side members extending along a length between opposite ends with a plurality of cross members extending between the second side members. Further yet, one of the opposite ends of the first member is configured for releasable fixed attachment to one of the ends of the second member. The other of the opposite ends of the first member is configured for releasable fixed attachment to a first support, and the other of the opposite ends of the second member is configured for releasable fixed attachment to a second support.

In accordance with another aspect of the invention, a patient support system in combination with a CT scanner having an annular opening through which a patient passes is provided. The combination includes a first support and a second support. In addition, the combination includes a radiolucent elongate first member having laterally spaced first side members extending along a length between opposite ends with a plurality of cross members extending between the first side members. Further, the combination includes an elongate second member having laterally spaced second side members extending along a length between opposite ends with a plurality of cross members extending between the second side members. One of the opposite ends of the first member is configured for releasable fixed attachment to one of the ends of the second member. The other of the opposite ends of the first member is configured for releasable fixed attachment to the first support, and the other of the opposite ends of the second member is configured for releasable fixed attachment to the second support. The CT scanner is configured to move between the first and second supports with the elongate first and second support members extending at least partially through the annular opening.

In accordance with another aspect of the invention, a method of installing a patient support system for a surgical procedure is provided. The method includes providing a radiolucent elongate first member having laterally spaced first side members extending along a length between opposite ends. Further, providing an elongate second member having laterally spaced second side members extending along a length between opposite ends. Further yet, providing a CT scanner having an annular opening with a first support on one side of the CT scanner and a second support on a side of the CT scanner opposite the first support. Then, coupling one of the opposite ends of the second member to the second support and coupling the end of the second member extended away from the second support to one of the ends of the first member. Further, coupling the end of the first member extended away from the second member to the first support.

### BRIEF DESCRIPTION OF THE DRAWINGS

These and other aspects, features and advantages of the present invention will become readily apparent when considered in connection with the following detailed description of presently preferred embodiments and best mode, appended claims and accompanying drawings, in which:

FIG. 1 is perspective view of a patient positioned on a patient support system constructed in accordance with one presently preferred aspect of the invention;

FIG. 2 is a view similar to FIG. 1 taken from a different perspective;

FIG. 3 is an end view of the patient support system of FIG. 1;

FIG. 4 is a top view of the patient support system of FIG. 1;

FIG. 5 is a side view of the patient support system of FIG. 1;

FIG. 6 is a perspective view of an assembled support surface of the patient support system shown disconnected from end supports of the system and being positioned on a mobile transportation and assembly cart therefor; and

FIG. 7 is a view similar to FIG. 6 showing the support surface in a disassembled state on the mobile transportation and assembly cart.

### DETAILED DESCRIPTION OF PRESENTLY PREFERRED EMBODIMENTS

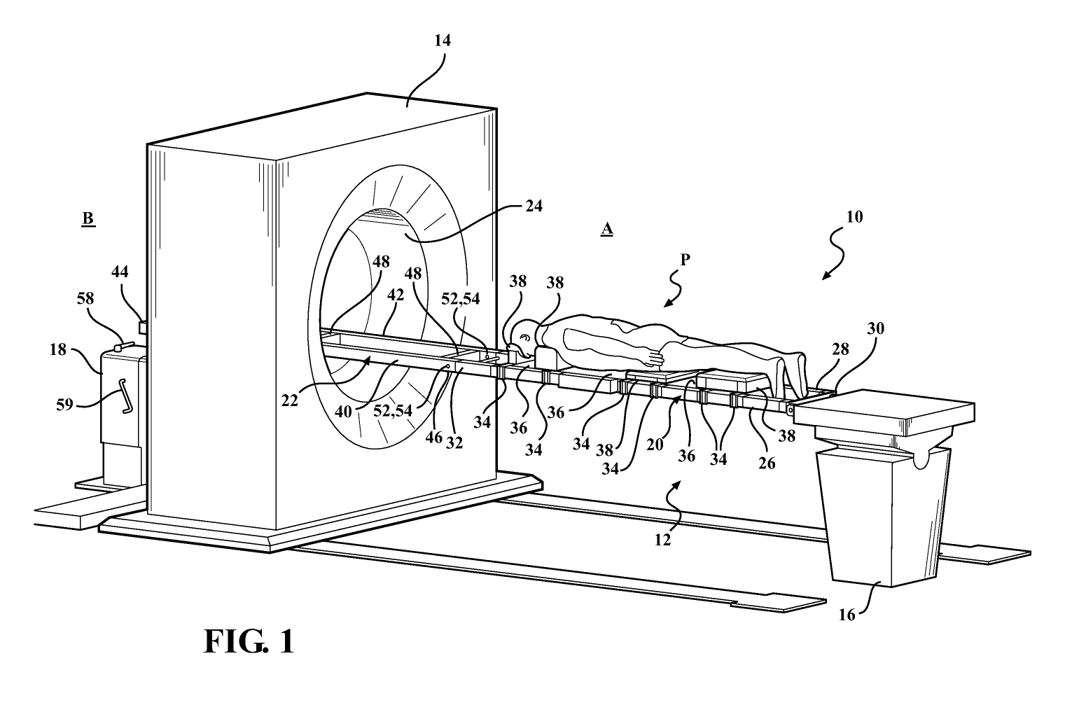

Referring in more detail to the drawings, FIG. 1 illustrates a patient support system, referred to hereafter as support system 10, constructed in accordance with one aspect of the invention. The support system 10 includes a support surface 12 that is configured to extend through a CT scanner 14; a first support 16 located on one side A of the CT scanner 14 and a second support 18 located on an opposite side B of the CT scanner 14. The support surface 12 includes a radiolucent elongate first member 20 and an elongate second member 22. The first member 20 is releasably attachable to the elongate second member 22 in such as way the first and second members 20, 22 remain rigid along all axes relative to one another. The first member 20 is releasably attached to the first support 16, while the second member 22 is releasably attached to the second support 18. A patient P is positioned on the first member 20, shown in a prone position, and during the surgical procedure, as desired, the CT scanner 14 is configured to move between the first and second supports 16, 18 along the full or substantially full length of the first member 20 with the first and second members 20, 22 extending at least partially through an annular opening 24 of the CT scanner 14. Accordingly, the CT scanner 14 is able to scan the entire or substantially entire body of the patient P during the surgical procedure.

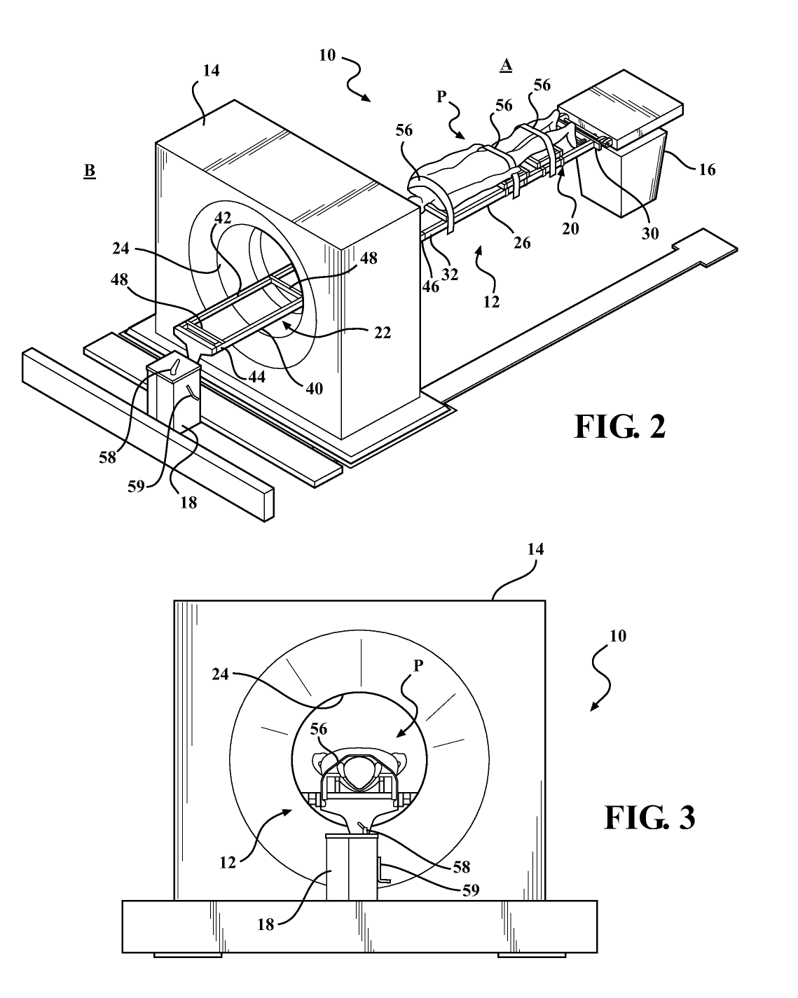

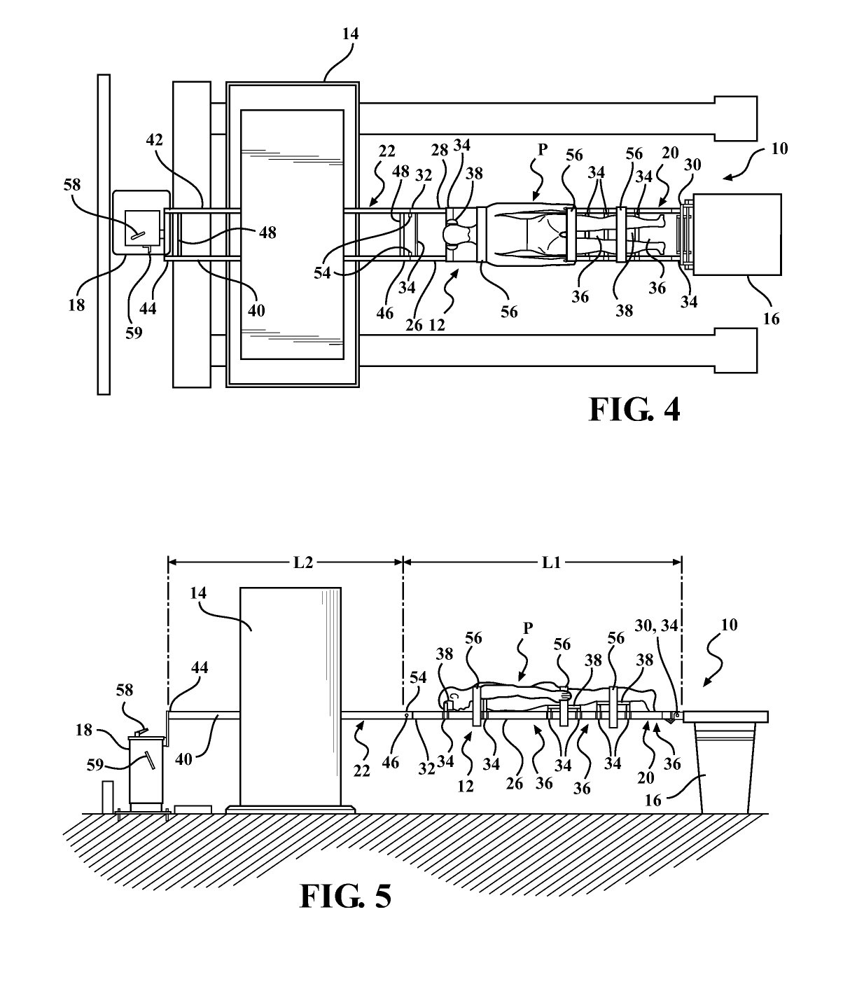

The first member 20 is constructed of radiolucent material, and can be constructed from carbon fiber, for example. The first member 20 has laterally spaced side rails, also referred to as first side members or sides 26, 28, extending along a length L1 (FIG. 5) between opposite ends 30, 32 (FIGS. 1, 2, 4-7) with a plurality of cross members 34 extending laterally between the sides 26, 28. The end 30 is configured for releasable attachment to the surgical table first support 16, such as any standard support that has lift and tilt capability. The other end 32 is configured for releasable attachment to the second member 22, as discussed further below. The cross members 34 function to make the first member 20 rigid, and further, at least some of the cross members 34 function to provide localized areas of support to the patient P. As such, the cross members 34 are strategically placed, wherein at least some are adjustable for movement and/or placement along the length L1 of the first member 20 to allow them to be positioned under selected areas the patient P, as desired. With the first member 20 having cross members 34, spaces 36 (FIGS. 1, 4 and 5) are provided along the first member 20 between the side rails 26, 28 and the various cross members 34. The spaces 36 allow unsupported areas of the patient P to extend between the side rails 26, 28, thereby minimizing the laterally cross-sectioned envelope of the patient P and the first member 20. As such, the first member 20 is ideally constructed to function as a spinal column support, wherein the CT scanner 14 is able to readily traverse the full body height of the patient P without interference, and further, provides the surgeon with unimpeded access to the patient P during surgery. The selected cross members 34 that are intended to provide support to the patient can be configured as straight or arcuate members as desired. Further, to facilitate positioning and providing comfort to the patient P, compliant, radiolucent support members 38 (identified in FIG. 1), e.g. pads constructed of rubber, foam or other types of elastic and/or compliant material, can be provided to extend between the first side members 26, 28 in fixed relation to selected cross members 34, as desired.

The second member 22 has laterally spaced side rails, also referred to as second side members or sides 40, 42, extending along a length L2 (FIG. 5) between opposite ends 44, 46 with a plurality of cross members 48 extending laterally between the sides 40, 42. The end 44 is configured for releasable attachment to the second support, also referred to as an end support 18, positioned on the side B of the CT scanner 14, wherein the end support 18 is configured to tilt and pivot in response to the commanded movement of the first support, also referred to as a surgical table support 16. The other end 46 is configured for releasable attachment to the end 32 of the first member 20 on the opposite side A of the CT scanner 14. The cross members 48 function to provide rigidity to the framework of the second member 22. The framework of the second member 22, not needing to be radiolucent, can be constructed from a radiopaque material, including metal, by way of example and without limitation, such as extruded aluminum and/or stainless steel, for example. Being extruded, the sides 40, 42 can be readily formed having a stiff, tubular form, thereby providing tubular channels in which the ends 32 of the first member 20 can be disposed. Of course, this is only one embodiment of many that achieve the same function. For example, it should be recognized that the side rails 40, 42 could be other than tubular, such as C-shaped or L-shaped in cross section, for example. To facilitate releasably fixing the first member 20 to the second member 22, an opening 52 can be formed though one or both sides walls of the second member 22 for receipt of a suitable fastener 54 (FIGS. 1, 4, 5 and 6), e.g. pin, bolt, spring detent, wherein the fasteners 54 can extend into a corresponding opening in the side rails 26, 28 of the first member 20.

During use, with the first member 20 being rigidly coupled to the second member 22, and with their respective ends 30, 44 coupled to the surgical table support 16 and the end support 18, respectively, the CT scanner 14 is positioned about the second member 22, with the second member 22 extending through the opening 24, while the surgeon performs surgery on the patient P. The surgeon is able to control the surgical table support 16 to lift and/or rotate the patient P as desired, while the support surface 12 moves conjointly in response to movement of the surgical support table 16. The end support 18 also allows the support surface 12 to move in conjunction with the support table 16 by using the manual height adjustment 59 and the pivot locking mechanism 58. Of course, the patient P is secured against movement on first member 20, such as by a plurality of straps 56. The straps 56 can be provide in any suitable location along the length L1 of the first member 20 to reliably secure the patient P in place. When movement of the support surface 12 is not desired, the end support 18 can be releasably locked via locking mechanism 58. When the surgeon wants to image the patient P, the CT scanner 14 is traversed along the support surface 12 in clearance relation therewith until the desired region of the patient P, and thus the first member 20, is received in the opening 24 of the scanner 14. As a result of how the patient P is positioned on the first member 20, at least partially between the side rails 26, 28, the entire head and torso region of the patient P can be readily imaged. Then, upon obtaining the desired image, the CT scanner 14 can be moved adjacent the end support 18, such that the entire first member 20 and patient P thereon is clear of the CT scanner 14.

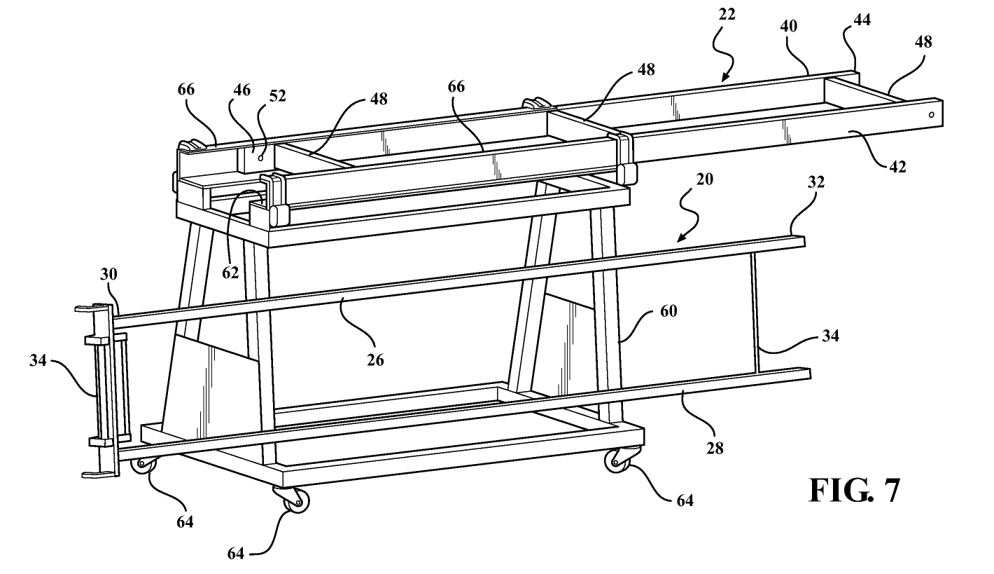

In accordance with another aspect of the invention, a method of using a patient support system 10 is provided. The method allows a support surface 12 and system 10 incorporating the support surface 12 to be installed in the operating room easily and efficiently by a single person. The support surface 12 is initially maintained in a broken down, reduced size state, with a first member 20 of the support surface 12 being decoupled from a second member 22 of the support surface 10. A portable cart 60 is configured to carry and transport the first and second members 20, 22 from a stowed position (FIG. 7) to the operating room. Though the second member 22 is shown releasably secured to an upper surface 62 of the cart in FIG. 7, it could be stowed along a side of the cart 60, similar to the first member 20. The cart 60 has castors 64 that allow it to be easily transported, as desired, wherein at least some of the castors 64 are lockable to prevent the cart 60 from unwanted movement. To initiate assembly, the cart 60, with the second member 22 releasably fixed on the upper surface 62, is moved to position and extend the end 44 of the second member 22 through the annular opening 24 of the CT scanner 14. Then, the end 44 of the second member 22 is coupled to the end support 18. Further, the first member 20 is then moved to bring its end 32 to rest on the surface 62 of the cart 60 with space being provided between the ends 32, 46 of the respective first and second members 20, 22. The end 30 of the first member 20, at this time, should be positioned to rest on top of the surgical table support 16. The first member 20 is then moved to bring its end 32 into attachment with the end 46 of the second member 22. Upon attaching the respective ends 32, 46 to one another, the opposite end 30 of the first member 20 is coupled to the surgical table support 16.

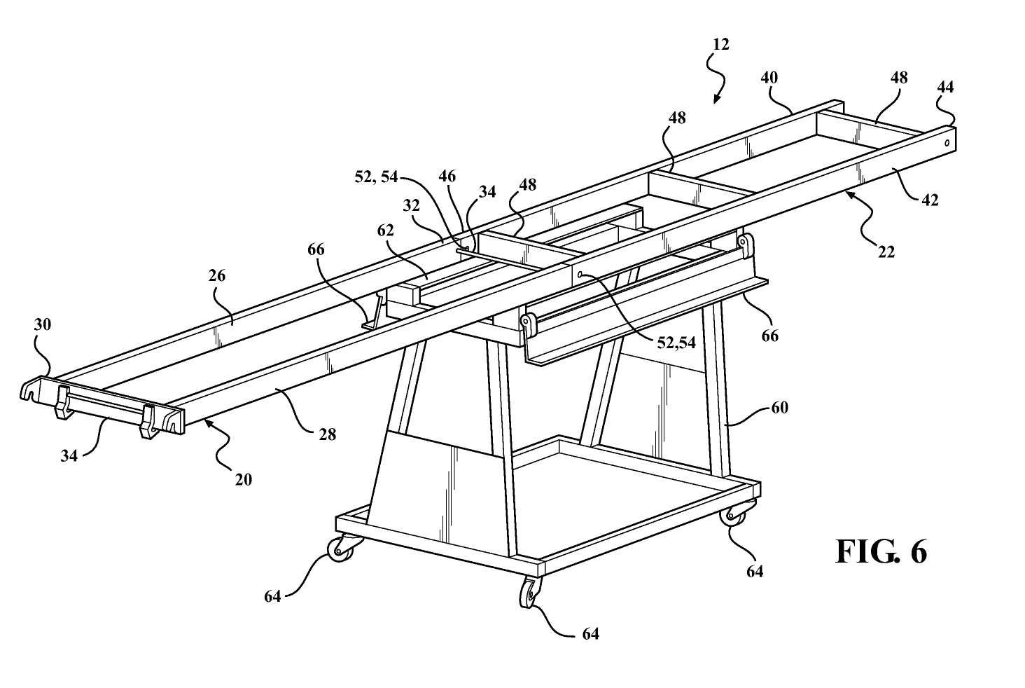

Upon assembling the ends 30, 44 of the support surface 12 to the surgical table support 16 and the end support 18, the cart 60 is removed from beneath the second member 22. This is done by releasing pivotal side clamps 66 from an upright clamped position (FIG. 7) to a downward unclamped position (FIG. 6). With the side clamps 66 pivoted to their downward unclamped position, the cart 60 is able to be rolled out from beneath the second member 22. As such, the installation of the support surface 12 is made easy, and can be readily performed by a single person.

Many modifications and variations of the present invention are possible in light of the above teachings. It is, therefore, to be understood that the invention may be practiced otherwise than as specifically described, and that the scope of the invention is defined by any ultimately allowed claims.

What is claimed is:

1. A patient support surface, comprising:
a radiolucent elongate first member having laterally spaced first side members extending along a length between opposite ends with a plurality of cross members extending between said first side members;
an elongate second member having laterally spaced second side members extending along a length between opposite ends with a plurality of cross members extending between said second side members; and
one of said opposite ends of said first member being configured for releasable attachment to one of said ends of said second member, the other of said opposite ends of said first member being configured for releasable attachment to a first support, and the other of said opposite ends of said second member being configured for releasable attachment to a second support.

2. The patient support surface of claim 1 wherein said second side members of said second member are radiopaque.

3. The patient support surface of claim 2 wherein said second side members of said second member are metal.

4. The patient support surface of claim 3 wherein said first member is constructed at least in part of carbon fiber.

5. The patient support surface of claim 1 wherein said first side members and said second side members are configured to extend in a clearance fit through an annular opening of a CT scanner.

6. The patient support surface of claim 1 further comprising a plurality of compliant, radiolucent support pads extending between said first side members.

7. A patient support system in combination with a CT scanner having an annular opening in which a patient is received, comprising:
a first support;
a second support;
a radiolucent elongate first member having laterally spaced first side members extending along a length between opposite ends with a plurality of cross members extending between said first side members;
an elongate second member having laterally spaced second side members extending along a length between opposite ends with a plurality of cross members extending between said second side members;
one of said opposite ends of said first member being configured for releasable fixed attachment to one of said ends of said second member, the other of said opposite ends of said first member being configured for releasable fixed attachment to said first support, and the other of said opposite ends of said second member being configured for releasable fixed attachment to said second support; and
said CT scanner being configured to move between said first and second supports with said elongate first and second support members extending at least partially through said annular opening.

8. The patient support system and CT scanner combination of claim 7 wherein said second side members are radiopaque.

9. The patient support system and CT scanner combination of claim 7 wherein said plurality of cross members extending between said second side members are radiopaque.

10. The patient support system and CT scanner combination of claim 8 wherein said second side members of said second member are metal.

11. The patient support system and CT scanner combination of claim 9 wherein said first member is constructed at least in part of carbon fiber.

12. The patient support system and CT scanner combination of claim 7 wherein said first side members and said second side members are configured to extend in a clearance fit through said annular opening of a CT scanner.

13. The patient support system and CT scanner combination of claim 7 further comprising a plurality of compliant, radiolucent support pads extending between said first side members.

14. A method of installing a patient support system for a surgical procedure, comprising:
providing a radiolucent elongate first member having laterally spaced first side members extending along a length between opposite ends;
providing an elongate second member having laterally spaced second side members extending along a length between opposite ends;
providing a CT scanner having an annular opening;
providing a first support on one side of the CT scanner;
providing a second support on a side of the CT scanner opposite the first support, the first and second supports being aligned with one another along an axis extending through the annular opening of the CT scanner;
coupling one of the opposite ends of the second member to the second support;
coupling the end of the second member extended away from the second support to one of the ends of the first member; and
coupling the end of the first member extended away from the second member to the first support.

15. The method of claim 14 further including performing the installation with a single person.

16. The method of claim 14 further including supporting the second member on a mobile cart while coupling one of the opposite ends of the second member to the second support.

17. The method of claim 16 further including removing the mobile cart from beneath the second support after coupling the end of the first member to the first support.
